(19) United States
(12) Patent Application Publication
(10) Pub. No.: US 2005/0142221 A1
(43) Pub. Date: Jun. 30, 2005

(54) Pharmaceutical activity and manufacturing method of the compound recipe product of 4 kinds of plants to increase testosterone

(76) Inventors: Jiao Gong, Arcadia, CA (US); Wei Jiang, Arcadia, CA (US)

Correspondence Address: James Chen, 150 N Santa Anita Ave., #560, Arcadia, CA, 91006, US

(21) Appl. No.: 10/745,807

(22) Filed: Dec. 29, 2003

(51) Int. Cl. A61K 35/78

(52) U.S. Cl. 424/725; 424/773

(57) ABSTRACT

The partial androgen deficiency of the aging male (PADAM) and the supplement of synthetic testosterone is an important research subject in the American biological science. The activity of leydigs cells of testis and the synthesizing and excreting state of testosterone has aroused wide concern of the medical circles. Taking synthetic testosterone may cause the cancer of genital system, which makes the middle-aged and old male people do not dare to taking the drugs of the synthetic testosterone. The Compound Recipe Product of 4 Kinds of Plants to Increase Testosterone is made by following procedures: Under the carefully and strictly controlled condition, use the technology of absorption and elution of WLD resin to extract and purify it and measured by the gas chromatograph and make a fingerprinting chromatograph quantitative determination so as to guarantee the product has a stable technological process and a high quality. The experiment of pharmacological activity shows that, by activating the leydigs cells of testis to enhance its ability to excrete testosterone, the compound recipe product has a remarkable function to obviously raise the quantity of testosterone blood serum of the normal and injured genital system and is used for treating the partial androgen deficiency of the aging male (PADAM) in clinical treatment.

### BACKGROUND OF THE INVENTION

The Pharmacological Activity and the Manufacturing Method of the Compound Recipe Product of 4 Kinds of Plants to Increase Testosterone belongs to the United States' patent of combination of the pharmacological activity of the natural products and the method to separate and purify the natural product. The product of 4 kinds of plants has the function to raise 17.44% of testosterone in blood serum of a normal rat. Especially, the product has function to obviously raise testosterone in blood serum of the genital system of the rat injured by cyclophosphane. In order to make more pharmacological activity of a compound to the public as more as possible, it is most important to encourage the research man. The Tax and Patent Appeals Court of United States considers the acknowledgement to the invention of chemical compound with pharmacological activity meets the requirements of the practicability of patent. The Pharmacological Activity and the Manufacturing Method of the Compound Recipe Product of 4 Kinds of Plants to Increase Testosterone conforms with the provisions made by the Tax and Patent Appeals Court of United States on the patent of pharmacological activity of chemical compound.

The androgen of male people is mainly produced by leydigscells of testis. The ovary and adrenal cortex of female people also secretes a small quantity of androgen. The testosterone is the most important androgen of male people. A normal female person produces about 0.25 mg of testosterone every day and a male person produces about 5-7 mg of testosterone every day. The research shows that there are 30-50% of androgen of male person is produced in the around tissue, the testosterone is produced in the cell, and a male person has active testosterone but not release extracelular space, which is a economical system that can have a maximal function with a minimal incretion. The general endocrine system needs a large quantity of incretion and only a small part of them are used for regulating and controlling and then come into degradation. Because the steroid receptor has high affinity, active androgen and estrogen is produced through activities of secretion in cell.

With increase of age, after climacteric, the generation of testosterone of androgen has a linear decline, which causes many symptoms of diseases such as psychological changes, reduction of sexual activities, reduction of density of skeleton, which affect the quality of life. Because taking the synthetic testosterone may causes cancers, there are some contentions about this issue. More people consider it should be done under the proper controlled condition to take testosterone as a complementary treatment. The testosterone reduces along with increase of age, which involves in the pathogenesis of diseases that related to the process of old age and the age. The testosterone is an important research subject in biological science in recent years. In America, the morbidity rate of male sexual hypoesthesia is 5%. The testosterone lack, which is one of most common disease of male persons, is caused by the injury of hypothalamus, hypophysis or testis. The purpose to supplement synthetic testosterone as a replacement therapy is to make the target tissue maintain the normal quantity of testosterone and its function, which only improves the quality of life but not prolongs one's life. The testosterone lack occurs at the adult stage, which can cause following symptoms: reduction of quantity of bones and osteoporosis, lower back pains, fracture of vertebra, baldness, if the testosterone lack lasts continuously, slight anemia, reduction of quantity of muscles and strength, atrophoderma, wrinkles, reduction of prostate volume, atrophy and softening of testis, and sexual hypoesthesia. How to supplement and increase the quantity of testosterone in body is a difficult problem of medicine that America medical circles want to solve but does not solve for a long time.

The Compound Recipe Product of 4 Kinds of Plants to Increase Testosterone presents a new scheme and method for solving the difficult problem.

### BRIEF SUMMARY OF THE INVENTION

In 1994, the Europe Andriatry Proseminar advanced the concept of “Partial androgen deficiency of the aging male, PADAM”.

The symptoms of PADAM is as follows: Nerve dysfunction, depression, hypomnesis, be absent-minded, fatigue, anypnia or lethargy, afternoon fever, hidrosis, irascibility, prostatic hyperplasia, sexual hypoesthesia. The specific classification of the symptoms is as follows:

The symptoms of nerve and blood vessel: Afternoon fever, hyperhidrosis, heart palpitation, and nervous temperament.

The symptoms of dysfunction of emotion and cognition: Dysphoria, bad self feeling, short of motive for life, reduction of brain function, short time hypomnesis, depression, short for self-confidence and fear without any reason.

The physiological and physical symptoms: Insomnia or lethargy, impaired appetite, constipation, atrophoderma, bones and joints pains.

The symptoms of virilism subsidence: Reduction of physical ability and strength, reduction of quantity and strength of muscles, pubisure falling off and abdominal corpulence.

The aging symptoms of urinary system: Frequent urinatin, urgent micturition, urinary incontinence, anuresis, not discharge all of urination, incapability of emiction, enuresis. Standard symptoms: feeling that urination is not discharged fully, still want to discharge urination within two hours after discharging urination, being intermittent when urination, urgency of micturition, thin urinary stream, difficulty of urination, more than two times of urination at night.

The symptoms of sexual hypofunction; Sexual hypoesthesia, obvious reduction or disappearance of long-time independent erection of penis, reduction of sexual activity, reduction of climax orgasm quality, ejaculation inability, oligospermia and sexual disfunction.

The concept of PADAM explains that the middle-aged person and old people indeed face the partial testosterone deficiency, which causes many kinds of disorders of sexual function, osteoporosis, atrophy and adynamia of the muscles, reduction of strength, and depression. In the past long period, medical authoritative persons ignored or not knew the important effect of factors of age on some diseases, which often resulted in the mistakes of basic conception of diagnosis and treatment and the severe side effect of drugs and complication.

Partial androgen deficiency of the aging male (PADAM) refers to the clinical state of partial reduction of the quantity of testosterone in body or reduction of sensitivity of organs to testosterone or active metabolic product. Hypogonadism causes the reduction of physical strength and the changes in the aspects of the health, sexual function and metabolism, which have the adverse impact on the functions of muscles, density of bone, fat metabolism and cognition, and cause many symptoms as followings: The vasomotion symptoms such as afternoonfever, idrosis, heart palpitation; The mental and psychological symptoms such as amnesia, anxiety; The physiological and physical symptoms such as insomnia, inappetence, bones and joints pains; The sexual symptoms such as reduction of sexual interest, sexual hypoesthesia, and erection dysfunction. All this is generally called as Partial androgen deficiency of the aging male (PADAM).

In June 2002, American Geriatrics Association reported that the research to the attention and memory of 300 old age people found that the people with higher testosterone has a better intellectual power of brain. The reduction of testosterone is the necessary cause of senile dementia. The senile dementia is the serious consequences of partial androgen deficiency of the aging male. The omen of senile dementia is lypemania. The patient has the following symptoms gradually: hypomnesis, more unresponsive than before, always forgetting things, selfish, being irritable, not allow others to touch his things, being wordy, and being unconcerned about his family members.

The medical terminology “partial androgen deficiency of the aging male (PADAM)” will replace the definition of traditional names of geratology such as male climacteric syndrome, menopause, menopause male active termination and etc. and it will become a formal and popular medical name of geratology in the world medicine. Because the technology of synthesizing testosterone and vicarious curative measures of the synthetic testosterone is stagnant and is detrimental to health, and also because America recently stopped using the vicarious curative measures of synthetic estradiol that has been used for fighting against female senium for more than 20 years, there will be a passive situation in which there will be short of proper medicine for treating this kind of disease in the world clinical medicine field. This great potential market is called as the greatest commercial opportunity with greatest potential in the new century. The estimated value of this commercial opportunity has exceeded the amount of sales of the software of Windows of Microsoft Company in the United States. Austria Urology Association points out that Partial androgen deficiency of the aging male (PADAM) recognized by the Europe Andriatry Association and the world medical circles does not clarify the following three medical theories:

The cause of disease of Partial androgen deficiency of the aging male (PADAM).

The relationship between Partial androgen deficiency of the aging male (PADAM) and prostatic hyperplasia

The relationship between Partial androgen deficiency of the aging male (PADAM) and the cause of disease of prostate and impotence

The difference between the Compound Recipe Product of 4 Kinds of Plants to Increase Testosterone and the synthetic testosterone is that the compound recipe product acts on the leydigs cells of testis and increase the secretory volume of testosterone by enhancing the activity of the cell. Another injury of taking synthetic testosterone is that it brings about the feedback inhibition function that will make the leydigs cells of testis, which has been in the state of disease, further lose its activity and become more aging. To sum up, we can find that it is a direct, simple, effective, safe, cheap and convenient treatment method to use the product of natural plants to stimulate the leydigs cells of testis to excrete afresh and continuously a great quantity of testosterone. The Compound Recipe Product of 4 Kinds of Plants to Increase Testosterone just is the classic medicine of this new kind of curative measures. Why does not the medical circles in the United States make use of this new medicine? Why to seek from afar what lies close at hand? Or why to abandon the easy way to seek a difficult way?

### BRIEF DESCRIPTION F THE SEVERAL VIEWS OF THE DRAWING

TABLE 1
The source of raw materials and the components
of the Compound Recipe Product of 4 Kinds of
Plants to Increase Testosterone

| | | |
|---|---|---|
| 1. | The above-ground branches and leaves of Herba Epimedii | 33% |
| | Berberidaceae | |
| | Epimedium brevicornum maxm. | |
| 2. | The succulent stem with scale leaf of Herba cistanchis | 20% |
| | Orobanchaceae | |
| | Cistanche deserticola Y. C. Ma | |
| 3. | The toot of Rhizoma Curculiginis | 20% |
| | Amaryllidaceae | |
| | Curculigo orchioides Gaertn. | |
| 4. | The toot of Radix Morindae officinalis | 27% |
| | Rubiaceae | |
| | Morinda offcinalis How | |
| | Total | 100% |

TABLE 2
The manufacturing method and the technological process of the
Compound Recipe Product of 4 Kinds of Plants to Increase Testosterone

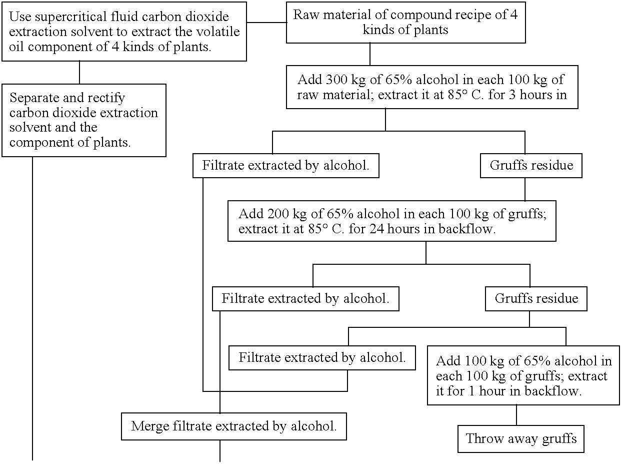

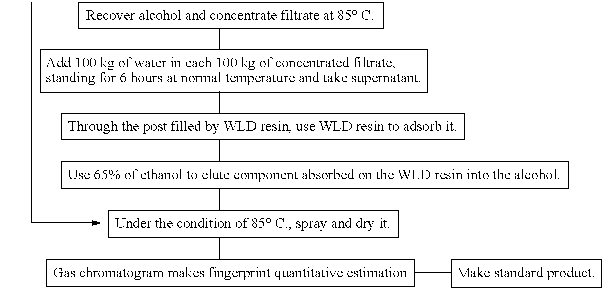

Table 3 The Pharmacological Activity of the Compound Recipe Product of 4 Kinds of Plants to Increase Testosterone

1. The effect of the Compound Recipe Product of 4 Kinds of Plants to Increase Testosterone (The product is shortly called as the compound recipe product.) on the testosterone in blood serum of the male rat.

The pharmacological activity of the Compound Recipe Product of 4 Kinds of Plants to Increase Testosterone has a close relationship with the quantity of testosterone in blood serum of body, luteinzing hormone and follicule-stimulating hormone. Using radioimmunity method to measure the content of testosterone in blood serum, we can understand whether the compound recipe product has the effect like androgen or not.

Taking the male rats, body weight: 290±40 g, divide them into groups at random. Pour distilled water for the rats of the First group, 20 ml/kg every day, pour the compound recipe product for the rats of the second group, 3.0 g/kg every day, this pouring continues for 28 days; in third and fourth groups, inject cyclophosphane in the abdominal cavity of rats, 20 mg/kg every day, this injection continues for five days, which produces the animal model with injured genital system. At same time, from first day, begin to pour distilled water and the compound recipe product for rats of third and fourth groups , which dosage and period of treatment is the same as the first and second groups. In the morning of the second day after stopping pouring the drug, kill the animal, separate blood serum according to the operational procedure of specifications of radioimmunoassay method of testosterone in blood serum, use Y-scintillation counter to measure radiate activity of sedimentary part of it, calculate the concentration of testosterone of every sample and gained the results by statistics.

The Result:

The compound recipe product of 4 kinds of plants can rises testosterone in blood serum of normal rat, but it has not significance in statistics; but the product of 4 kinds of plants can rise obviously the quantity of testosterone in blood serum of rats which genital system are injured by cyclophosphane. (See Table 1.)

The effect of compound recipe product on testosterone
in blood serum of male rats

| | Number of | Testosterone in |
|---|---|---|
| | Animal | Blood Serum |
| Group | (Piece) | (ng/dl, x ± SD) |
| The Distilled Water Group | 6 | 152.44 ± 147.85 |
| The Compound Recipe Product Group | 6 | 179.04 ± 155.89* |
| The Cyclophosphane + | 6 | 91.48 ± 34.59** |
| Distilled Water Group | | |
| The Cyclophosphane + | 6 | 143.83 ± 56.47ΔΔ |
| Compound Recipe | | |
| Product Group | | |

### DETAILED DESCRIPTION OF THE INVENTION

1. The pharmacological activity and Manufacturing method of the Compound Recipe Product of 4 Kinds of Plants to Increase Testosterone belongs to the United States' patent of combination of the pharmacological activity of natural product and the method to separate and purify natural product. The product of 4 kinds of plants contains the following four kinds of products: Herba Epimedii 33%, Herba Cistamchis 20%, Rhizoma Curculiginis 20%, Radix Morindae Officinalis 27%.

2. After using carbon dioxide to extract the raw materials of 4 kinds of plants, separate and rectify the extracted liquid and make it as a reserved material, and then respectively add 300 kg, 200 kg and 100 kg of 65% of alcohol in each 100 kg of raw material of 4 kinds of plants, extract it for 3, 2, 1 hour in backflow under the condition of 85° C., merge filtrate extracted by alcohol, recover alcohol and concentrate filtrate under the condition of 85° C., and then add 100 kg of water in each 100 kg of concentrated filtrate, standing for 6 hours at normal temperature and take supernatant. Then, through the post filled by WLD resin, use WLD resin to absorb, use 65% of ethanol to elute the component absorbed on the WLD resin into the alcohol, under the condition of 85° C., spray and dry it. After gas chromatogram makes fingerprint quantitative estimation, make the standard product.

3. The result in experiment on the effect of Compound Recipe Product of 4 Kinds of Plants to Increase Testosterone on the testosterone in blood serum of the male rat shows that the compound recipe product can raise the testosterone in blood serum of the normal rat, and the raised rate is 17.44% higher than the control group. And the product can obviously raise the quantity of testosterone in blood serum of the rat which genital system is injured by cyclophosphane (See Table 3.).

1. I declare that the pharmacological activity and Manufacturing method of the Compound Recipe Product of 4 Kinds of Plants to Increase Testosterone, which consists of following products: Herba Epimedii, Herba Cistamchis, Rhizoma Curculiginis, Radix Morindae Officinalis, is my invention.

2. I consider that my invention contains the following process: Use supercritical fluid carbon dioxide extraction solvent to extract and rectify the compound recipe raw materials of 4 kinds of plants, after abstracted by ethanol and treated by water, use WLD resin to adsorb, separate and purify it and dry the liquid, and finally make the standard product after measuring the fixed quantity of component. Compound recipe product.

3. I consider that in my invention, the compound recipe product of 4 kinds of plants has the pharmacological activity to obviously raise the quantity of testosterone blood serum of normal and injured genital system, and can be used for treating the partial androgen deficiency of the aging male (PADAM).
